(19) United States
(12) Patent Application Publication
(10) Pub. No.: US 2013/0052725 A1
(43) Pub. Date: Feb. 28, 2013

(54) SYSTEM FOR OPTICAL BASED DELIVERY OF EXOGENOUS MOLECULES TO CELLS

(71) Applicants: Siavash Yazdanfar, Niskayuna, NY (US); Vasile Bogdan Neculaes, Niskayuna, NY (US); Kenneth Roger Conway, Clifton Park, NY (US); Allen Lawrence Garner, Clifton Park, NY (US); Evelina Roxana Loghin, Rexford, NY (US); Dmitry Vladimirovich Dylov, Schenectady, NY (US); Brian Michael Davis, Albany, NY (US); Chulmin Joo, Niskayuna, NY (US)

(72) Inventors: Siavash Yazdanfar, Niskayuna, NY (US); Vasile Bogdan Neculaes, Niskayuna, NY (US); Kenneth Roger Conway, Clifton Park, NY (US); Allen Lawrence Garner, Clifton Park, NY (US); Evelina Roxana Loghin, Rexford, NY (US); Dmitry Vladimirovich Dylov, Schenectady, NY (US); Brian Michael Davis, Albany, NY (US); Chulmin Joo, Niskayuna, NY (US)

(73) Assignee: GENERAL ELECTRIC COMPANY, SCHENECTADY, NY (US)

(21) Appl. No.: 13/285,976

(22) Filed: Oct. 31, 2011

Related U.S. Application Data
(63) Continuation-in-part of application No. 13/221,161, filed on Aug. 30, 2011.

(51) Int. Cl. C12M 1/42

(52) U.S. Cl. 435/289.1

(57) ABSTRACT

A system for delivering exogenous molecules comprises a support for containing cells and exogenous molecules; an infra-red (IR) light source that generates an IR optical beam with an average power density at least greater than 10⁵ W/cm²; one or more optical elements; an imaging system to image the cells in a field of view; a processor that generates a signal for localization of cells in the field of view; a light pattern shaper for temporal focusing of optical beam to generate wide field illumination on the cells to permeabilise the cell membrane for delivering the exogenous molecules; and a controller that switches optical beam from wide field illumination to a focused illumination. The processor is operatively coupled to the imaging system and the light pattern shaper and transmits the signal for the localization of cells to ensure the temporal focusing of the optical beam on the cells.

### CROSS REFERENCE TO RELATED APPLICATIONS

This application is a continuation-in-part of U.S. patent application Ser. No. 13/221,161, entitled “Optical Based Delivery of Exogenous Molecules to Cells”, filed Aug. 30, 2011; which is herein incorporated by reference.

### TECHNICAL FIELD

The invention relates generally to systems for delivering foreign materials into living cells using optical means, and more specifically, the invention relates to systems for delivering exogenous molecules into living cells using infrared (IR) light.

### BACKGROUND OF THE INVENTION

The introduction of exogenous molecules into cells plays a significant role in recombinant technology, therapeutic applications, genetic analysis, cell tracking or cell trafficking. For example, delivery of genetic materials into cells is a key technique in recombinant DNA technology and has immense importance in genetic analysis. In transfection, the cells are administered with naked DNA, which are introduced into the cells to produce an RNA and/or protein.

A successful delivery of exogenous molecules into cells in an in vitro system can be achieved by various techniques to improve efficiency of the delivery technique without perturbing the structure or function of the cells. One or more techniques are known in the art to introduce exogenous molecules into the cells which include, but are not limited to, microinjection, liposome based cell fusion, electroporation, and ballistic methods. Moreover, pulsed electric field and diagnostic ultrasound are presently used for permeabilization of cells. Among these techniques, electroporation has been used for various types of cell transfection, though it is largely disruptive and causes cellular death.

To increase the efficiency and to minimize the side effects of various methods or systems, optical-based delivery was introduced. For example, laser based microinjection, optoinjection or optoporation are used for gene delivery. Use of lasers for cell transfection is an efficient method, and a hole formed by the laser beam is found to repair itself within a short time span. In optoporation, either permeability of the cell membrane is changed or pore formed in the plasma membrane, and these techniques do not damage the cells extensively. In most of the optoporation techniques, the permeability of the cells is modified at the site at which the laser impacts the cell membrane. However, the methods or the systems used for laser based microinjection, optoinjection or optoporation presently allow for low throughput because they are generally focused on single cell transfection. Additionally, these methods and the systems used are expensive, employing high numerical aperture objectives and light sources (e.g., solid state lasers).

Therefore, optical based delivery techniques and systems for delivery, which are inexpensive and provide high throughput molecular delivery into cells with high transfection efficiency are desirable.

### BRIEF DESCRIPTION OF THE INVENTION

One or more of the systems of the invention overcome many of the disadvantages of the delivery systems known in the art. One or more of the examples of the systems of the invention are able to transfect exogenous molecules to the cells without affecting cell viability in an inexpensive way.

In one embodiment, a system for delivering exogenous molecules into one or more cells having a cell membrane, comprises a support for containing the cells and exogenous molecules, an infra-red (IR) light source that generates an IR optical beam with an average power density of at least 10⁵ W/cm² and one or more focusing elements, an imaging device, a processor that generates a signal corresponding to a localization of cells, and a light pattern shaper that is configured to temporally and spatially shape the optical beam on the cells for a period of time adapted to permeabilise the cell membrane, wherein the processor is operatively coupled to the imaging device and the light pattern shaper and transmits the localization signal to the light pattern shaper.

In one embodiment, a system for delivering exogenous molecules into one or more cells having a cell membrane, comprises a support for containing the cells and exogenous molecules, wherein the support is a microfluidic chip, an infra-red (IR) light source that generates an IR optical beam with an average power density of at least 10⁵ W/cm² and one or more focusing elements, an imaging device, and a processor that generates a signal corresponding to a localization of cells, wherein the processor is operatively coupled to the imaging device and transmits the localization signal to the system.

### BRIEF DESCRIPTION OF THE DRAWINGS

These and other features of embodiments of the invention will be more readily understood from the following detailed description of the various aspects of the invention taken in conjunction with the accompanying drawings that depict various embodiments of the invention, in which:

FIG. 1 is a schematic drawing of a system for optical based delivery of exogenous molecules, according to one embodiment of the invention.

FIG. 2 is a schematic drawing of a system for optical based delivery of exogenous molecules with microfluidic chip, according to one embodiment of the invention.

### DETAILED DESCRIPTION OF THE INVENTION

Optical based delivery systems enable the exogenous molecules to be delivered into the cells without compromising the viability of the cells, cellular expansion, death, and differentiation. The system is configured to deliver exogenous molecules to cells by adding exogenous molecules to the cells, exposing the cells to an IR light source and permeabilising the membrane of the exposed cells.

To more clearly and concisely describe and point out the subject matter of the claimed invention, the following definitions are provided for specific terms, which are used in the following description and the appended claims. Throughout the specification, exemplification of specific terms should be considered as non-limiting examples.

As used herein, “area of irradiation” refers to an area over which a defocused IR radiation is incident. The cells are typically present within that area and consequently are being irradiated by the IR light. The cells are exposed to IR over the area of irradiation.

As used herein, “exogenous molecules” refers to the molecules which are not present or derived outside the cells. The exogenous molecules may be transferred to the cells from outside. The exogenous molecules may comprise genetic materials (such as DNA or RNA), proteins, peptides, drugs, or small molecules such as dyes.

As used herein, “thermal gradient” refers to a rate of temperature change with distance. For example, a temperature difference between the inside and outside of the cell provides a thermal gradient across the cell membrane. The thermal gradient may be generated based on light absorption of the water molecules present in the liquid media (comprising the cells) and lipid molecules of the cell membrane.

As used herein, “target sequence” refers to a sequence that codes for an expression product. In some examples, the expression product may be able to elicit an immune response in an organism. The target sequence codes for a surface antigen of the bacterium, virus, fungus, parasitic organism, or non-parasitic organism from which it is derived.

As used herein, “vertical adjustment of the laser focusing” refers to an adjustment method that positions the optical focus along the optical axis where the laser intensity is modified by vertically shifting one or more optical components (such as, lenses or mirrors) towards or away from a support (such as sample holder). Therefore, vertical adjustment refers to an adjustment of the optical elements along the optical axis. Vertical adjustment may be necessary to control the efficiency of delivery of exogenous molecules, such as transfection, or ablation control for a particular cell type. In some applications, this can be accomplished manually, with automatic translation stages, variable focus lenses and wave front modulators.

As used herein, “high throughput” refers to a method for delivery of exogenous molecules to multiple cells at a time. The number of cells for high throughput delivery is generally more than 10. In some examples, the high throughput number may be 10⁵ or more. For example, in high throughput cell transfection, the number of cells transfected in one example is in a range from about 20 to 30. The high-throughput delivery typically employs delivery of exogenous molecules to multiple cells by irradiating the cells simultaneously, per exposure. One or more of robotic, data processor, controller, detectors, and related software may be used to achieve high throughput delivery.

As used herein, “temporal focusing” refers to a method of illumination of an object with a pulsed laser, where an optical dispersion is adjusted such that the laser pulses incident on the object in a short temporal duration. For example, the duration of laser pulses is in an order of 100 femtoseconds emitted from a laser source, can be broadened by material dispersion as the laser beam travels through lenses and other optical elements. The pulse broadening can be reversed by use of appropriate dispersive elements, positioned such that pulses arriving at the object are close to 100 femtoseconds in duration. In another embodiment, the role of the optical element providing temporal dispersion may be played by diffraction grating.

As used herein, “operatively coupled” refers to a connection, which may be direct or indirect. The connection is not necessarily being a mechanical attachment. For example, in a system comprising one or more devices, wherein the devices may be coupled to each other such that there is no direct coupling when the system is in non-operational state. However, at the time of operating the system, the devices are coupled to each other to make the system functional.

As used herein, “average power density” is a ratio of absolute power to the illumination area. The peak power density may be estimated from average power density and multiplying it by a ratio T/t, where T is a period of pulse repetition and t is duration of each laser pulse.

A system for delivering exogenous molecules into one or more cells having a cell membrane, comprises a support for containing the cells and exogenous molecules, an infra-red (IR) light source that generates an IR optical beam with an average power density of at least 10⁵ W/cm² and one or more focusing elements, an imaging device, a processor that generates a signal corresponding to a localization of cells, and a light pattern shaper that is configured to temporally and spatially shape the optical beam on the cells for a period of time adapted to permeabilise the cell membrane, wherein the processor is operatively coupled to the imaging device and the light pattern shaper, and transmits the localization signal to the light pattern shaper.

The support for containing cells and exogenous molecules is interchangeably used herein as a “sample holder”. Therefore, the sample holder is used for holding exogenous molecules and a plurality of cells. Typically, the cells are placed on the holder of the system, wherein the cells are irradiated by IR optical beam to permeabilize the cell-membrane. In one embodiment, the area of irradiation is an area corresponding to the sample holder, whereas in another embodiment the area of irradiation partially covers the sample holder. The sample (cells) is mounted on the sample holder, wherein the position of the sample holder is adjustable to optimize focusing or defocusing of light on the sample. In one embodiment of the system, the sample holder is adjustable for altering the position of the cells with respect to the optical beam with required illumination intensity. By adjusting the vertical position of the sample holder, the focus of the optical beam on the holder may be changed. In one embodiment, the sample holder is placed in a position, so that the cells are placed at the focus of the optical beam. In another embodiment, the sample holder is placed in a position, so that the cells are placed in a defocussed region (or out of focus) of the optical beam to irradiate multiple cells. In this embodiment, the cell-sample may be lifted to a certain position by lifting the sample holder, to place the sample in the defocused part of the optical beam. In these embodiments, the movement of the sample holder may be controlled via a lever, switch, or an automated electronic motion controller and the position of the holder may be changed depending on its requirement. For selected delivery of exogenous molecules to multiple cells, the use of defocused optical beam is desirable.

In one or more embodiments of the system, the support for containing cells and exogenous molecules is a microfluidic chip. In some embodiments, the support is a multi-channel microfluidic chip. The microfluidic chips may be purchased commercially or fabricated using standard materials (e.g., PDMS, SU-8, etc.) and procedures (e.g., lithography). The chip comprises one or more inlet ports for insertion of cells to the support or sample holder of the system. The chip further comprises one or more outlet ports for collection of the cells after optical based delivery of exogenous molecules to the cells. A hydrodynamic force is generated inside the fluidic channel to drive the cells in the direction of the inlet port to the outlet port, and passes through the IR optical beam generated by the IR optical source of the system. The cells are entered into the microfluidic channels with a steady flow towards the outlet, and are in constant motion while traversing through the support of the system. The dimensions of the chip suits particular application and may vary from sub-millimeters to centimeters, with channel diameters of around 100 nanometer to several hundred micrometer.

To maintain a steady fluid flow at the time of optical based delivery, one or more pumps are used to regulate and control the flow. One pump may be used for inserting the fluid into the channel, and another pump may be used to collect the fluid out from the channels. In some embodiments, one pump may be used to drive the fluid stream comprising cells, and another pump may be used to drive fluid stream of buffer. Flow rate of the fluid is controlled by two pumps and may set in accordance with time required for delivery of particular molecules, such as from seconds to tens of minutes depending on application, cell type, and molecular size. For example, flow rate may set to be higher than 1 μL/min for sample and buffer. The flow rate of the cell depends on the confluency of the cells, viscosity of the cell medium, and the size of the molecules. In some embodiments, the microfluidic chip and fluids are sterilized before each experiment. The conduits used for the system including syringes or connecting tubes are washed with 70% ethanol, followed by washing with filtered, sterilized, deionized water and dried before use.

The system may be configured to permeate cell membrane and deliver exogenous molecules into the cells by varying focus of the IR optical beam. Various focusing elements are used to change the focus of the optical beam, such as variable-focus liquid crystal lens, spatial modulator and wave front modulator to move optical focus rapidly. In some examples of the method, using different focusing lenses, the focal point of the optical beam may be changed. In one example, a variable-focus liquid crystal lens may be used to change the focal point of the optical beam. In another example, translation of a lens along the optical axis may be used to change the focal point of the optical beam.

In some embodiments, the focal position of the laser relative to the cell membrane may be controlled using appropriate focusing elements. Varying the focus of the optical beam using various focusing elements and changing the position of the support for containing cells and exogenous molecules, the cells are positioned at the defocused region of the optical beam for optical based delivery. Therefore, the exogenous molecules are delivered to multiple cells by using the defocused optical beam. In some embodiments, the optical beam is temporally focused for wide field illumination to irradiate the cells. Wide field illumination occurs when the optical energy is spread among a large number of cells, in contrast to a localized illumination where only a few cells are illuminated. In one or more embodiments, the system is configured to place the sample holder in a relatively defocused region of the optical beam for delivering exogenous molecules. The cells are placed at the focal point (focus) of the optical beam in the case of cell ablation. In some embodiments, a combination of localized and wide-field illumination techniques may also be used. The system is configured to be flexible to do a single cell transfection depending on its requirement, while the system is also configured to transfect a large number of cells by wide field illumination for transfecting multiple cells at once.

The number of cells that are irradiated depends on the density of the cells at the time of exposure and on the area of exposure. If the density of the cells is high, the number of cells exposed to the IR optical beam is greater than the number of cells exposed in the case of lower cell density. In some embodiments, the area of irradiation, which is the area irradiated by defocused region of the IR optical beam, is about 50 to 200 μm². In one embodiment, the area of irradiation of the IR optical beam is about 100 to 200 μm². Using large field of view, the defocused region of the IR optical beam can irradiate up to 1000 μm².

In one or more embodiments, the system comprises a controller that switches the optical beam from the wide field illumination to a focused illumination. In this embodiment, the exogenous molecules are transferred to a single cell, as the area of irradiation is much smaller than the area in case of a defocused beam. In some embodiments, a focused illumination or a wide field illumination occurs independently using the controller as per user requirement. For example, the controller is configured to generate focused illumination which is useful for ablating cells, when the average power density of the optical beam is higher than the beam used for optical based delivery. The user may select various options, such as focused illumination, defocused illumination, average power density of the IR beam, size of the cell sample, and amount of exogenous molecules required for delivery and may provide command to the controller to select respective parameters for the selected method. For example, a user may use a touch screen, a switch, or a remote to select individual parameters or groups of parameters for optical based delivery.

In one or more embodiments of the system comprises the infra-red (IR) light source that generates an optical beam with an average power density of greater than or equal to 10⁴ W/cm² at the optical focus and a peak power density that is greater than or equal to an order of 10⁸ W/cm². The system further comprises one or more optical elements, and an imaging system to image the cells in a field of view. In some embodiments of the system, the IR light source generates the optical beam, which is generally in a wavelength range between 0.7-3 μm and is used for optical based delivery. In one or more embodiments of the system, the IR light source may be a laser with a center wavelength of approximately 1.55 μm. In one or more embodiments, IR optical source of the system has an average optical power of at least 50 mW. In some embodiments, the IR source is used with an average optical power of about 100 to 500 mW. In one embodiment, the IR source has an average optical power in a range from about 100 to 200 mW.

Strong IR absorption by water present in the media comprising cells, results in heating effect. The use of IR light with high average power density results in cell damage due to heating effect on irradiation. Therefore, the use of an IR source which generates less average power density is desirable to increase the cell viability. Use of a defocused low power IR light for multiple times over a population of cells enables higher efficiency for delivery of exogenous molecules with minimal cell death.

The system is configured to generate a pulsed optical beam, which may be used to suppress the heat generation on the cell membrane and may consequently minimize the cell damage. In some embodiments, the IR light source is a pulsed light source, such as laser or laser diode. Therefore, in these embodiments, the IR optical beam is generated through the pulsed light source. In one embodiment, the IR optical beam that is used for delivery of exogenous molecules is a laser beam. In one or more embodiments, the IR light source generates a laser beam after passing through one or more of the variable-focus liquid crystal lens, spatial modulator or wave front modulator and by moving optical focus rapidly. By reducing the intensity of the laser beam, optimizing pulse duration of the beam, minimizing the average power of the beam, or using a broader area of irradiation, the viability of the cells may be increased. For example, the area of irradiation in this example is a broader area of about 50 to 200 μm², wherein the produced heat may be diffused on the multiple cells, and reduces the damage of the irradiated cells.

The laser beam generated by the system has a good directionality, and may be focused or defocused depending on its requirement using an optical microscope. These properties enable the laser beam to increase permeability of the cell membrane by irradiating the cells. Various types of laser may include, but are not limited to, the YAG laser, the eximer laser, the Ar ion laser, the nitrogen laser, or the nitrogen-excited color laser. One or more embodiments of the system operate in the longer wavelengths, and the light source may be fiber-based, which enables compact implementation of the delivery method. In one embodiment, an erbium-doped fiber laser is used to produce a defocussed laser beam for irradiating the cells. In one example, the system employs the erbium-doped fiber laser at a wavelength of 1.55 μm, unlike other expensive and large solid-state lasers, e.g., Nd:YAG or Ti: Sapphire laser.

In some embodiments of the system, the IR light source is configured to generate a lower average power density and peak power density at the optical focus, which are suitable for optical based delivery of exogenous molecules with minimum cell damage. As noted, the average power density is a ratio of absolute power to the illumination area, and the peak power density may be calculated from average power density and multiplying it by a ratio T/t, where T is a period of pulse repetition and t is duration of each laser pulse. In one example, the period of pulse repetition T is 20 ns, duration of each laser pulse t is 100 fs. As noted, the system is configured to generate a lower average power density of the IR light at the optical focus, such as 10⁴ W/cm², unlike other methods presently used for optical based delivery that use a much higher average power density. In some embodiments of the system, the IR light source is configured to generate an average power density of the IR light at the optical focus that is greater than or equal to 10⁴ W/cm², and a peak power density that is greater than or equal to 10⁸ W/cm². In one or more embodiments, the average power density of light generated at the optical focus is in a range of 10⁴ to 10⁵ W/cm². In one or more embodiments, the average power density of IR light generated at the optical focus is in a range of 10⁵ to 10⁶ W/cm². In these embodiments, the peak power density is in a range of about 10⁸ to 10⁹ W/cm².

In some embodiments of the system, the IR source generates an optical beam that produces a thermal gradient across the cell membrane to irradiate the cells for permeabilization, unlike other nonlinear absorption and cascade ionization processes employing expensive high-power ultrafast lasers at visible or near IR spectral range. A short wave infrared (SWIR) pulsed lasers with wavelengths of 1˜3 μm induces the thermal gradient, where water absorption is significantly higher than other wavelengths employed in conventional optoporation methods. The optical beam with low power density illuminates multiple cells in a single-shot using inexpensive fiber-based laser. In some embodiments, the IR optical beam comprises short wavelength IR (SWIR) with a wavelength in a range of about 1.4 to 3 μm.

The system comprises a light pattern shaper. The light pattern shaper is used for regulating temporal and spatial properties of the optical beam. For shaping the temporal properties of the optical beam, the pattern shaper comprises a diffractive element such as a diffraction grating or prism. A pulsed light source illuminates the diffractive element, and the diffracted beam is then projected by a lens system to the sample holder. The signal of localization of cells in a particular position of the sample holder transmits to the light pattern shaper for generating temporally focused beam in the field of view to ensure the temporal focusing of the optical beam on the cells. In one embodiment, the light pattern shaper comprises a diffraction grating and one or more lenses. The determined pattern of the optical beam is transferred into the light pattern shaper at the time of operating the system. In embodiments for shaping the spatial properties of the optical beam, the light pattern shaper may create a patterned illumination of the IR light to allow targeted delivery of exogenous molecules. For example, the light pattern shaper is composed of digital micro-mirror based system (DMD). A DMD uses a mirror array to pattern the light, thereby illuminating only the target region. Other variants for light patterning methods may be utilized, for example, with spatial light modulator or holographic methods. The light pattern shaper may include elements for both temporal and spatial shaping. The temporal focusing unit is typically composed of diffraction grating and a 4-F imaging system to ensure that temporally focused pulsed light may be formed only on the cell membranes. The light pattern shaper changes the transfection beam pattern accordingly for selected cell transfection. A unique aspect in this embodiment is to use refractive or diffractive lenses for detection and transfection of cells. For example, a Fresnel lens can be manufactured with a small form factor (<100 μm) and a high numerical aperture (NA), which improves transfection efficiency. Typically, temporal focusing of light is used for positioning the shortest pulse at a desired location along with the beam propagation path. Temporal focusing is achieved by illuminating a diffraction grating at particular angle, called a Bragg angle, and collecting diffracted light at the sample by a focusing lens. In one embodiment of the system, the Bragg angle is equal to 30°. Temporal focusing ensures that the wide field laser illumination is arriving at the samples, wherein the laser illumination is comprised of pulses that are compressed in time, and thus have high peak intensity, in order to achieve high thermal gradients. The temporal focusing generates a uniform gradient of temperature, which increases efficiency of optical based delivery, for example optical based transfection.

In some embodiments, the multiple laser pulses are generated through the system to irradiate the cells, wherein the laser beam has a lower intensity. The high efficiency of laser-based optical delivery may be achieved using repeated sublethal laser pulses without affecting cell-viability. In one embodiment, the laser pulse has a wavelength greater than at least 700 nm. In some embodiments, the width of each of the laser pulses is less than about 500 fsec. In some other embodiments, the width of each of the laser pulses is less than about 200 fsec. In another embodiment, the pulse width of the laser used for the method is about 100 fsec. The varied number of laser pulses is incident upon the cell membrane with a repetition rate of at least 1 MHz in one example, and the repetition rate of the pulses is at least 10 MHz in another example. In some embodiments, the repetition rate of the pulses is at least greater than 50 MHz.

In some embodiments, the optical based delivery system module is integrated with an imaging system, a biomedical imaging system, or an image analyzer. The integration of an optical based delivery system with an imaging system improves viewing, analysis, and selection of cells prior to the delivery process. It also improves the efficiency of the delivery process. The optical based delivery system may be a compact module, which is easy to integrate with another system, such as an imaging system. In this embodiment, the imaging system may be a large system, where the optical system is fitted at the appropriate position. In one or more embodiments, the optical system may be integrated into a conventional microscope. For example, the optical system may be integrated into a fluorescence or phase contrast microscope. Hence, the integrated system comprises the functions of optical imaging and optical transfection.

The processor receives images from the microscope and selects cells within regions of the image to be illuminated for delivery. The processor may select or exclude cells by automated segmentation or pattern recognition algorithms. If a variety of cell types are present in the sample holder, the algorithms may be used to select a subset of cells for delivery. Different cell types may include cells from the same source but at different stages within the cell cycle. For example, the processor may choose to avoid cells that are undergoing mitosis at the time of delivery.

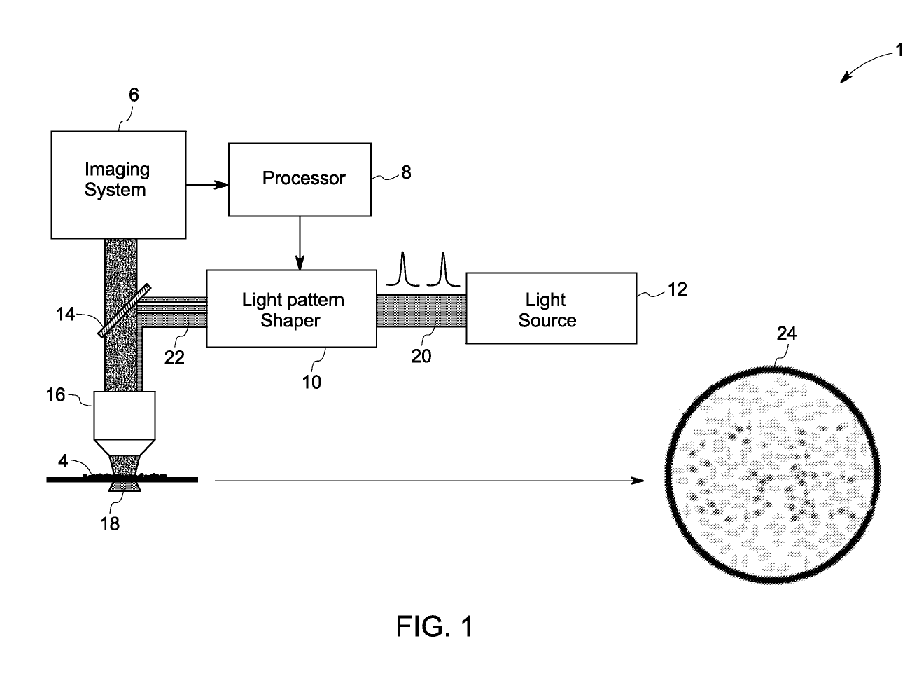

FIG. 1 shows an exemplary system 2 for high-throughput optical based delivery, for a cell transfection system. In FIG. 1, the system 2 comprises a holder or cell support comprising cell sample 4, wherein the imaging system 6 first captures the images of cells based on the structural or molecular contrast. The image information is fed into a processor 8 by detecting and locating the target cells in a field of view (FOV) and the processor determines optimal strategies for spatial pattern of the transfection light in the FOV. The determined pattern is transferred into the light pattern shaper 10. The optical beam 20, which generates from the light source 12, then passing through the light pattern shaper 10 and converts to the patterned beam 22. The light pattern shaper 10, which is, for example, composed of digital micro-mirror based system (DMD) and temporal focusing unit. A DMD spreads the transfection light over a mirror array, for example a dichroic mirror 14, and patterns the light by directing only the light to the target region and passes through one or more optical elements 16 before incident on the cells. The optical beam, after passing through various modules and optical elements, is incident on the cells, wherein the cells are exposed to a defocused part of the beam 18 for wide field illumination. Other variants for light patterning methods may be utilized, for example, with spatial light modulator and holographic methods. The temporal focusing unit is typically composed of a diffraction grating and a 4-F imaging system to ensure that temporally focused pulsed light may be formed only on the cell membranes. Using system 2, targeted delivery 24 of the exogenous molecules to the cells is shown.

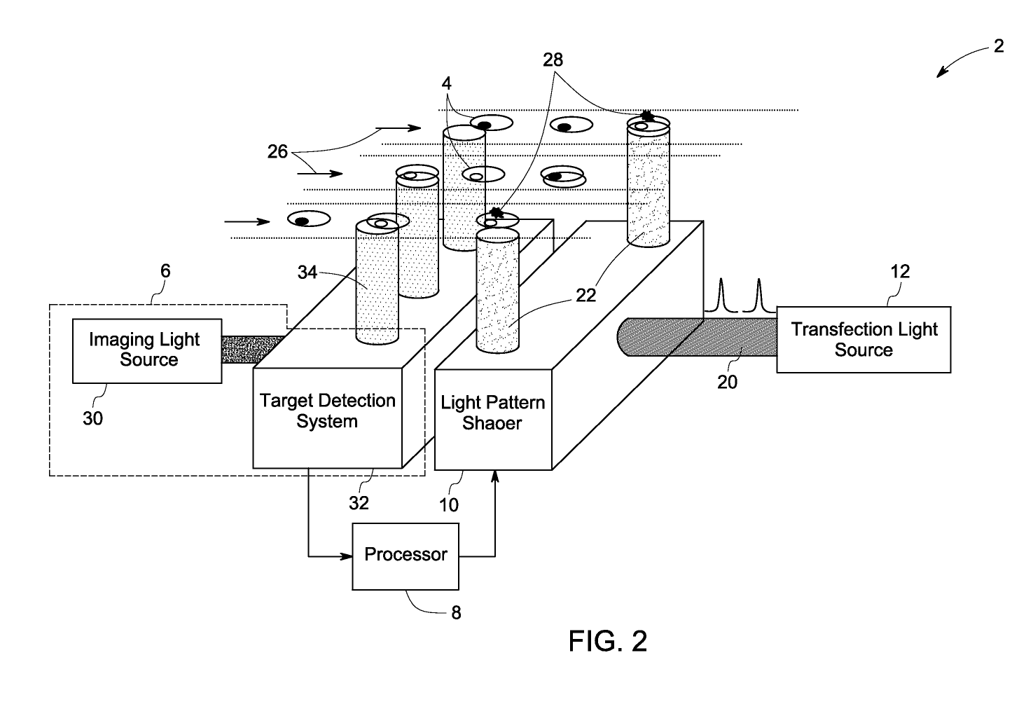

A second exemplary embodiment of the system 34 is illustrated in FIG. 2. A multi-channel parallel microfluidic unit 26 is used for high-throughput cell transfection. Different types of cells flow through the channels, wherein cellular signatures such as fluorescence or scattering are detected through target detection system 32, which is a part of the imaging system 6. The imaging system also comprises the imaging source 30. The processor receives this signal and determines the channels to be illuminated for transfection. The light pattern shaper 10 changes the transfection beam pattern accordingly for selected cell transfection. A unique aspect in this embodiment is to use refractive or diffractive lenses 28 for detection and transfection of cells.

At the time of operating the system, the time of exposure to a laser beam may be controlled, so that the exposure may be enough for making the cell membrane permeable, and at the same time the exposure may not reach a threshold for killing the cells. For effective optical-based delivery, a beam residence time for irradiation is important. In some examples, the exposure of the cells may be between about 1 to 30 seconds, which is typically enough to permeabilize the cell membrane. The exposure of cells may be even higher for few minutes, depending on the cell type or light source, for example 1 to 5 minutes, wherein the cell viability is maintained. In one example, the beam residence time is about 10 minutes, and the cells are viable after this prolonged exposure, as the intensity of the light is optimized to a lower value.

The permeabilization of cells results by producing sub-micrometer holes on the membrane to facilitate uptake of exogenous materials into the cells. The permeabilization is the result of absorption of multi-photon and cascade ionization of water molecules on the cell membranes. For efficient delivery of exogenous molecules into the cells, a thermal gradient may be generated across the cell membrane of each of the cells, which may permeabilise the cell membrane temporarily. The thermal gradient is the results of excitation of the water molecules present in the liquid media comprising the cells. Unlike other optical delivery that uses visible and NIR light (300 nm˜1064 nm), the method employs an IR light source with longer wavelengths (1˜10 μm), which have higher water absorption. The higher water absorption results in a larger thermal gradient in the sample in the IR than in the visible. The IR light encompasses the near infrared (700 nm to 1.4 μm), shortwave infrared (1.4 μm to 3 μm), mid-infrared (3 μm to 8 μm) and portions of the long infrared (8 μm to 10 μm). In one example, water molecules absorb light at a wavelength of about 1.55 μm to generate light induced microscopic thermal gradient across the cell membrane. The lipid molecules present on the membrane also play a significant role in inducing the thermal gradient.

In some embodiments, greatly differing absorption coefficients between the water of the liquid media comprising the cells and the lipid of the cell membrane, play an important role. Differing absorption coefficients affects the thermal gradient across the cell membrane. In one aspect, a large thermal gradient facilitates the cell membrane poration process. In another aspect, the exogenous molecules may move from a high temperature environment to a low temperature environment.

As shown in table 1, conventional optical based delivery methods generally use wavelengths of about 0.532 μm, wherein the absorption coefficient of water is 0.000447 cm⁻¹ and that of lipid is 0.01002 cm⁻¹ and the ratio of water:lipid is about 0.045. Some of the conventional methods use optical spectra with a wavelength of about 0.80 μm, wherein the absorption coefficient of water is 0.02 cm⁻¹ and that of lipid is 0.004 cm⁻¹ and the ratio of water:lipid is about 5. In some examples of the methods, the wavelength used for delivering exogenous molecules is about 1.55 μm, wherein the absorption coefficient of water is 10.5 cm⁻¹ and that of lipid is 0.08 cm⁻¹ and the ratio of water:lipid is about 131.25. The optical based delivery at the wavelength of 1.55 μm provides at least an order of magnitude higher difference of absorption coefficients between water and lipid, and the ratio of ˜131.25, which is almost about 2900 times higher than the ratio for delivery at 0.532 μm and 26 times higher than the ratio for delivery at 0.80 μm. As can be seen in Table 1, the required power density at the optical focus is dependent on the wavelength of the light source and the absorption coefficient of the sample at said wavelength. Specifically, higher absorption coefficients require lower power densities at the sample to achieve the same level of optical delivery. For example, moving further into the IR beyond 1.55 μm, absorption coefficient of water is higher in orders of magnitude. This allows the power density at the optical focus to be adjusted accordingly.

TABLE 1
Ratio of absorption coefficients of water and lipid at
different wavelengths.

| | | Wavelength (μm) | | |
|---|---|---|---|---|
| | | 0.532 | 0.80 | 1.55 |
| Absorption | Water | 0.000447 | 0.02 | 10.5 |
| coefficient | Lipid | 0.01002 | 0.004 | 0.08 |
| (1/cm) | Water/lipid | 0.045 | 5 | 131.25 |

In one or more embodiments, the optical based delivery system is used for transfection. Genetic materials may be delivered to the cells using the laser exposure. Use of pulse laser is even more beneficial, as the pulse laser with lower intensity does not affect the cell morphology or cellular-function. As noted in this embodiment, the use of defocused laser on a broader area using the system enables transfection of more than one cell at a time.

In one embodiment, the system allows molecular delivery of multiple cells simultaneously, such as transfection of multiple cells. The illumination pattern of the near IR optical beam enables multiple cells to uptake exogenous molecules. In one or more embodiments, the number of cells transfected by this example is at least in a range from about 20 to 30 or more. In one embodiment, the system allows a high throughput transfection of multiple cells. The high-throughput delivery typically employs delivery of exogenous molecules to multiple cells by irradiating the cells, per exposure. For achieving an automated high throughput delivery, use of one or more of robotic, data processor, controller, detectors, and related software may be desirable. In one embodiment, by changing illumination pattern of the near IR light by suing light pattern shaper, optical delivery of exogenous molecules to single cell may also be possible.

As noted in the optical based delivery, the exogenous molecules are transferred from outside to the cells, wherein the cells may be selected from all type of cells including, for example bacterial cells, yeast cells, plant cells or animal cells. The efficiency of optical based delivery is not dependent on cell types, unlike other chemical transfection methods. In the case of plant cells, the cell wall may be removed, for example, using any conventional method. The spheroplast forms after removing the cell-wall, and may be used for cell transfection using optical based delivery. The cells may be placed in a static or moving condition. In one example, the cells may be placed on a holder, wherein the cells are in static condition. In another example, the cells may be moved through a channel while illuminating by laser for optical based delivery of exogenous molecules.

In the optical based delivery using the system, the exogenous molecules which are delivered to the cells may comprise many materials such as, small molecules, genetic materials, proteins, peptides, organelle, physiologically active materials, and targeting agents. The genetic material comprises deoxyribonucleic acids (DNA), ribonucleic acids (RNA), small interfering ribonucleic acids (siRNAs), micro RNA, nucleic acid analogues, oligonucleotides, plasmids, and chromosomes.

In some embodiments, the genetic material used is DNA. The DNA may be a double-stranded DNA (dsDNA), single-stranded DNA (ssDNA), plasmid DNA, synthetic DNA, organelle DNA or circular DNA. In one example, the DNA has a recombination site. In some embodiments, the exogenous molecules are DNA, wherein the DNA has a target sequence. The target sequence may code for an expression product. In some examples, the target sequence may code for an expression product, which is capable of eliciting an immune response in an organism. In one or more examples, the target sequence may be derived from at least one of: a bacterium, virus, fungus, parasitic organism and non-parasitic organism. In some examples, the target sequence codes for a surface antigen of the bacterium, virus, fungus, parasitic organism or non-parasitic organism from which the target sequence is derived. An illustrative use of an optical based delivery is to express recombinant proteins, such as the proteins which are able to generate a signal (e.g. expression of a green fluorescent protein) and that is useful in tracking cells (e.g. during cell therapy). The exogenous molecules may be used to elicit an immune response in an organism.

In one or more examples, the exogenous molecule is short interfering RNA or siRNA. RNA interference (RNAi) is a natural antiviral defense mechanism for silencing gene expression and presently is a powerful technology to knockdown genes in mammalian cells for functional analysis or gene-therapy. siRNA is useful for inhibiting the expression of a gene of interest, such as a gene with a defect results a phenotypic, a genotypic or both effect. Typically, siRNA is intracellular double stranded RNA that regulates post transcriptional gene silencing pathways. Using siRNA techniques, specific gene function may be determined through targeted gene knockdown. An antiviral response may be overcome by using delivery of siRNA as exogenous molecules. The optical based delivery may directly be introduced siRNA into the cytoplasm through the temporarily permeable membrane, without forming endosome. Therefore, the delivery of siRNA using the optical based method may bypass the endosomal processing pathways. In one example, the genetic material may be micro RNA.

In one embodiment, the exogenous molecules are coupled to one or more agents. The agents may be selected from a bead, an affinity tag, a signal peptide, a nanoparticle, a vesicle, a lipid and combinations thereof. A bead or affinity tag may be coupled to the exogenous molecules. For example, when glutathione-S-transferase (GST) tag sequence is coupled to DNA, the recombinant DNA enables isolation and purification of a protein encoded by the same DNA using respective affinity column. For another example, a signal peptide may be coupled to a genetic molecule, such as a nuclear localization signal or NLS is coupled to DNA, wherein the recombinant DNA enters into a specific location of the cell, such as nucleus, using the NLS signal sequence.

One of the optical based delivery systems utilizes engineered nanoparticles that produce shock waves or localized heat upon light illumination. The system may be performed targeted transfection by attaching engineered nanoparticles to the target cells. For example, metallic nanoparticles may be engineered for attaching the nanoparticle to the membrane of the target cells, and to generate high thermal gradient upon light absorption. In one embodiment, carbon nanoparticles such as carbon nanotubes, carbon black, or graphene generates significant sound waves upon light absorption, which facilitates molecular uptake by the cells. In another embodiment of the system, the engineered nanoparticles may be suspended in the liquid media where the cells are placed, and the nanoparticles are used to generate significant sound waves that may have an additional effect, which helps uptaking exogenous molecules into the cells.

In one or more embodiments, the system is configured to optical based delivery, wherein the optical based delivery may also combine with other delivery methods. Such as, the laser exposure may be associated with treating the cells with one or more chemicals to increase the permeability of the cells. The system may comprise a provision for pre-treatment, simultaneous treatment or post-treatment of the cells. The treatments of cells may take place in the system itself, or may take place outside the system. For example, magneto-transfection or magneto-poration may be used before the laser exposure. In this embodiment, the magnetic force may align the cells, exogenous molecules, or combination of both at the optical focus, wherein the exogenous molecules are subsequently be delivered to the cells using the laser exposure. In some other examples, the permeability of the cell membrane may increase using various chemicals having mild effect on the cell membrane, and then the exogenous molecules are subsequently be delivered to the cells using laser beam. For example, use of calcium phosphate, cationic polymer such as polyethylene imine, or liposomes enables the cell membrane to uptake exogenous molecules, and the cells which are processed with such agents may easily be transfected with much higher efficiency.

Cell ablation may be required in some of the applications, such as, to prevent undesired stem cell differentiation, or to resist unlimited cell growth in different cellular pathways, such as apoptosis. Uncontrolled cell growth is a feature of malignancy, which may also be resisted using malignant cell ablation using laser treatment. The laser has an excellent light-condensing ability, and contributes no thermal influence upon a portion other than the area of irradiation of the laser beam. Depending on the vertical adjustment of the laser focusing, the intensity of the laser may be changed. The cells, which are required to be ablated, are generally placed at the focus of the laser beam. In case of optical based delivery, the cells are typically placed at the region which is out of focus for the laser beam. In one or more examples, the intensity of the laser may increase to an extent which is enough for ablating the cells. The beam residence time may also vary depending on the requirement of the application. In one example, the laser beam residence time or exposure time is optimized in combination with appropriate average power density of the laser, which may be sufficient to ablate or kill the cells. Similar efficiency may be achieved either by increasing the beam residence time while the power density of the laser is low, or by keeping the beam residence time small, while the power density of the laser is high. In some examples, the beam residence time and the power density of the laser may need to be adjusted at the same time for optimizing the optical based delivery process.

The system for delivery of the exogenous molecules into the cells has efficiencies comparable with the systems commonly used. Small molecules, such as PI, calcein, dextran, phalloidin, and macromolecules, such as DNA, RNA and siRNA are successfully delivered to the cells using laser exposure, in accordance with one embodiment of the invention.

The scope of the invention is defined by the claims, and may comprise other examples not specifically described that would occur to those skilled in the art. Such other examples are intended to be within the scope of the claims.

What is claimed is:

1. A system for delivering exogenous molecules into one or more cells having a cell membrane, comprising:
a support for containing the cells and exogenous molecules;
an infra-red (IR) light source that generates an IR optical beam with an average power density of at least 10⁵ W/cm² and one or more focusing elements;
an imaging device;
a processor that generates a signal corresponding to a localization of cells; and
a light pattern shaper that is configured to temporally and spatially shape the optical beam on the cells for a period of time adapted to permeabilise the cell membrane,
wherein the processor is operatively coupled to the imaging device and the light pattern shaper and transmits the localization signal to the light pattern shaper.

2. The system of claim 1, wherein the focusing elements are configured to generate a wide field illumination.

3. The system of claim 2, wherein the focusing elements are configured to illuminate an area of 50 to 1000 μm².

4. The system of claim 1, wherein the support is a multi-channel microfluidic chip.

5. The system of claim 1, wherein the focusing elements comprise refractive and diffractive lenses.

6. The system of claim 1, wherein the light pattern shaper comprises a digital micro-mirror based device (DMD) and a temporal focusing unit.

7. The system of claim 6, wherein the temporal focusing unit further comprises one or more diffraction gratings.

8. The system of claim 1, wherein the imaging device comprises a fluorescence microscope, bright field microscope, phase contrast microscope or a combination thereof.

9. The system of claim 1, wherein the IR light source is a short wave infrared (SWIR) pulsed laser.

10. The system of claim 9, wherein the SWIR pulsed laser generates a wavelength in a range from about 1 to 3 μm.

11. The system of claim 10, wherein the SWIR pulsed laser generates a wavelength greater than 700 nm, a pulse width less than 1000 psec, and a repetition rate greater than 10 MHz.

12. The system of claim 10, wherein the SWIR pulsed laser is a fiber laser.

13. The system of claim 1, wherein the support has a position relative to the optical elements and the support is configured to adjust the relative position either manually or automatically.

14. The system of claim 1, wherein the light pattern shaper is configured to change an illumination pattern of the optical beam.

15. The system of claim 1, wherein the optical elements are configured to make a vertical adjustment of temporal focusing.

16. The system of claim 15, wherein the cells are positioned at the defocused region of the optical beam.

17. The system of claim 1, further comprising one or more elements for changing the beam from a wide field illumination to a focused illumination.

18. The system of claim 1, wherein the IR light source is configured to generate a thermal gradient across the cell membrane.

19. A system for delivering exogenous molecules into one or more cells having a cell membrane, comprising:
a support for containing the cells and exogenous molecules, wherein the support is a microfluidic chip;
an infra-red (IR) light source that generates an IR optical beam with an average power density in an order of at least greater than 10⁵ W/cm² and one or more focusing elements;
an imaging device; and
a processor that generates a signal corresponding to a localization of cells;
wherein the processor is operatively coupled to the imaging device and transmits the localization signal to the system.

20. The system of claim 19, further comprising a light pattern shaper that configured to temporally and spatially shape the optical beam on the cells for a period of time adapted to permeabilise the cell membrane.

21. The system of claim 19, further comprising one or more elements for changing the optical beam from a wide field illumination to a focused illumination
